(19) United States
(12) Patent Application Publication
(10) Pub. No.: US 2006/0089621 A1
(43) Pub. Date: Apr. 27, 2006

(54) Bone mill and template

(76) Inventor: Mike Fard, Charlottesville, VA (US)

Correspondence Address: Whitham, Curtis & Christofferson, PC, Suite 340, 11491 Sunset Hills Road, Reston, VA, 20190, US

(21) Appl. No.: 10/803,189

(22) Filed: Mar. 18, 2004

(51) Int. Cl. A61B 17/00

(52) U.S. Cl. 606/1

(57) ABSTRACT

Bone mills and templates for use in during bone resectioning is provided. The templates have a flat upper surface or an interior track, and the mill has an edge that rides on top of the upper surface or within the groove. The template thus guides the mill during bone resectioning, delimiting the area and depth of the bone that is removed. Additional bone mills with supports and frames that control the depth of milling are also provided.

### BACKGROUND OF THE INVENTION

1. Field of the Invention

The invention generally relates to a mill and template for use during bone resectioning. In particular, the invention provides a template with an interior track that guides the mill during resectioning of the bone.

2. Background of the Invention

Knee “replacement” surgery is becoming more and more common as a result of increased longevity and the attendant increase in geriatric related diseases such as osteoarthritis. The term “replacement” is a misnomer in that the entire knee is not replaced. Rather, diseased portions of the tibial and femoral condyles of the knee are removed and replaced with endoprosthetic (metal and/or polymer) inserts. While such replacement surgery is a vast improvement over the prospect of pain and immobility due to a diseased knee, the operation is non-trivial, typically requiring a 10-12 inch incision, extensive resectioning of the bone, and weeks or months of rehabilitation.

An alternative which may be suitable for some patients is unicompartmental (unichondylar) replacement. In this case, one of two compartments of the knee (medial or lateral) is targeted for resurfacing and replacement with endoprostheses. This is frequently the medial compartment due to the way weight is distributed during walking. The unicompartmental procedure is much less invasive, typically requiring only a 3-4 inch incision, much less bone resectioning, and a shortened time of rehabilitation.

During knee surgery, the surgeon must remove worn and damaged surfaces of the tibia (shin bone) and the femur (thigh bone) where they articulate in the knee. Small segments of healthy bone must also be removed in order to provide a suitable surface for mounting the prosthetic implants, and it is desirable to remove the least amount of bone possible. FIG. 1A schematically illustrates a typical prior art procedure, showing a knee joint with femur 10 having had bone segment 11 removed by resectioning to create femoral space 12, and tibia 20 having had bone segment 21 removed to create tibial space 22. FIG. 1B shows insertion of prosthetic femoral implant 13 into femoral space 12, and insertion of tibial prosthetic implant 23 into tibial space 22. For simplicity of illustration, in FIG. 1, the bone segments that are removed are depicted as a single, discrete L-shaped piece of bone. In reality, the bone may be removed in small segments or slices by sawing or milling, and the final shaping of the space may be carried out by milling to achieve a level surface with dimensions suitable for attachment of the prosthesis.

Despite significant advances in the technology that supports such osteosurgery, the result of such an operation is surprisingly dependent on the individual skill of the surgeon, since the final steps of shaping the bone require “free-hand” milling of the bone surface and a trial and error approach to finally fitting the implant onto the bone. While some technologies do exist for guiding the cutting and milling of the bone to ensure a correct fit of the prostheses and alignment of the knee bones, many involve very elaborate mechanical devices that are expensive and very complicated to operate. For example, U.S. Pat. Nos. 5,344,423 and 5,486,180 to Dietz et al. describes an apparatus for milling bone that includes a template with a reference surface for controlling the depth of a cut and a track for guiding the cutter in two dimensions to cut a planar surface. However, the template comprises two portions, one of which is movable and thus relatively complex, and which causes the template to take up additional space in a cutting area where space is very limited. U.S. Pat. No. 5,908,424 to Bertin et al. and US design patent to Dietz provide a template for determining the extent of milling of a bone in two dimensions, and designs for the template, respectively. However, in this case the depth of milling is controlled by a relatively complex system involving a separate attachment that serves as a depth monitor. U.S. Pat. No. 5,474,559 to Bertin et al. provides femoral milling instrumentation which is suitable for total knee arthroplasty, and which is comprised of relatively complex multiple slots for establishing s series of reference planes on the bone to be milled. U.S. Pat. No. 5,601,563 to Burke et al., is directed to a milling guide with a detachable cutting guide. The milling guide does not include a means for controlling the depth of the milling. Such technologies do not provide a simple system for controlling both the two dimensional boundaries and the depth of milling. They do little to decrease the time required for carrying out the milling procedure, and may be so complex as to dissuade surgeons from attempting their use.

The prior art has thus far failed to supply technology that allows accurate, three-dimensional milling of bone surfaces to a desired size and shape in a straightforward, accurate and affordable manner.

### SUMMARY OF THE INVENTION

The present invention provides templates and milling devices for milling bone to a desired, standardized size and shape. The invention allows a surgeon to accurately remove a volume of bone of a defined, three-dimensional shape, thereby creating a space in or on the bone for placement of an endoprosthetic device that fits the space. The templates of the present invention are used as a guide to limit the extent of bone removal by a milling device, i.e. to delimit (set the boundaries of) the depth, lateral dimensions and shape of the volume of bone that is removed. Further, use of the templates and milling devices of the present invention allows the removal of less bone than in known, prior art procedures.

It is an object of this invention to provide a template for bone milling. In one embodiment, the template comprises a frame having a top, a bottom, one or more external sidewalls, and one or more internal sidewalls. The frame has one or more openings extending there through from the top to the bottom, and at least one of said one or more internal side walls defines a peripheral boundary of each of the one or more openings. The template also comprises a guide track formed in the one or more internal sidewalls, the guide track receiving a guide of a bone milling device whereby the bone milling device may be moved about the peripheral boundary using the guide track.

The template may further include a means for removably securing the frame to a bone which is to be milled. In one embodiment, the means for removably securing includes one or more tabs projecting from the frame which have one or more securing points which may be secured to a bone. The one or more tabs may project from the one or more external sidewalls.

In a preferred embodiment, the guide track is positioned approximately midway between the top and said bottom of the frame, and has a flat lower surface which is approximately parallel to the top and said bottom of the frame. In one embodiment, the guide track has an angled upper surface which projects at an angle from the lower surface to a point relatively closer to the top of the frame than to the bottom of the frame. Alternatively, the guide track may have an arcuate upper surface which extends from the lower surface to a point relatively closer to the top of said frame than the bottom of the frame. In yet another embodiment, the guide track has an arcuate lower surface and an angled upper surface which projects at an angle from the lower surface to a point relatively closer to the top of the frame than to the bottom of the frame.

In an alternative preferred embodiment, the template lacks a guide track rather, the bone milling tool includes a region specifically designed to rest on the surface of the template and to abut against the inside peripheral wall of the template. This configuration sets the depth of milling while atht eh same time restricts the milling to a defined area.

In some embodiments, the frame of the template is curved to match one or more curves of a bone. For example, the frame may have a peripheral boundary in a shape configured to accommodate a femoral implant or a tibial implant. Further, the peripheral boundary of the template may have one or more bulbous regions. These bulbous regions permit the milling device to mill out the bone to match the peripheral corners of the insert.

The present invention also provides a kit for partial knee replacement surgery. The kit preferably includes: i) a plurality of tibial implants; ii) a plurality of tibial frames, each of the tibial frames having a top, a bottom, an external sidewall, and an internal sidewall, and each of the tibial frames having an opening extending therethrough from the top to the bottom of the tibial frame, wherein the internal side wall defines a peripheral boundary of the opening, and each of the plurality of tibial frames has an opening sized to match one of the plurality of tibial implants; iii) in one embodiment, a guide track formed in the internal sidewall of each of the tibial frames, the guide track receiving a guide of a bone milling device whereby the bone milling device may be moved about the peripheral boundary using the guide track; iv) at least one femoral implant; and v) either at least one femoral frame having a top, a bottom, an external sidewall, and an internal sidewall, the at least one femoral frame having an opening extending therethrough from the top to the bottom of the femoral frame wherein the internal side wall defines a peripheral boundary of the opening, and wherein the opening is sized to match one of the at least one femoral implants, or a took specifically designed to mill the femur. In one embodiment, the femoral frame and the femoral implant are curved to match at least one curve of a femur bone. The kit may also include a bone milling tool which either fits within the guide track of the tibial frame, or which has a region that rest on top of the tibial frame and abuts against an inner peripheral sidewall of the frame.

The tibial and femoral implants may be constructed from, for example, metal, plastic or ceramics.

The kit may further include means for removably securing each of the tibial and femoral frames to a tibia or femur bone, respectively. In one embodiment, the means for removably securing includes one or more tabs projecting from the tibial or femoral frame, and have one or more securing points which may be secured to a bone. The one or more tabs project may from the external side wall of the frame. In different embodiments, the tabs may bo on one side of the frame or on opposite sides. A hook mechanism may also be secured to the frames for holding the frame securely to the tibia during milling, but which can be unhooked after milling.

In a preferred embodiment, the guide track in each of the tibial and femoral frames is positioned approximately midway between the top and said bottom of the frame. The guide track may have a flat lower surface which is approximately parallel to the top and the bottom of the frame. In some embodiments, the guide track has an angled upper surface which projects at an angle from the lower surface to a point relatively closer to the top of the frame than to the bottom of the frame. In other embodiments, the guide track has an arcuate upper surface which extends from the lower surface to a point relatively closer to the top of the frame than to the bottom of the frame. In yet other embodiments, the guide track has an arcuate lower surface and an angled upper surface which projects at an angle from the lower surface to a point relatively closer to the top of the frame than to the bottom of the frame. Further, the peripheral boundary of at least one of the plurality of tibial implants has one or more bulbous regions.

The kit may further include a guide track formed in the internal sidewall of the at least one femoral frame, the guide track receiving a guide of a bone milling device whereby the bone milling device may be moved about the peripheral boundary using the guide track. In a preferred embodiment, the guide track is positioned approximately midway between the top and bottom of the frame. The guide track may further have a flat lower surface which is approximately parallel to the top and bottom of the frame. The guide track may have an angled upper surface which projects at an angle from the lower surface to a point relatively closer to the top of the frame than to the bottom of the frame. In yet another embodiment, the guide track has an arcuate upper surface which extends from the lower surface to a point relatively closer to the top of the frame than to the bottom of the frame. Alternatively, the guide track may have an arcuate lower surface and an angled upper surface which projects at an angle from the lower surface to a point relatively closer to the top of the frame than to the bottom of the frame.

In a preferred embodiment, the kit also includes a bone milling device. The bone milling device is preferably a one time use disposable device. The bone milling device preferably has a milling bit which is angled from a drive member, preferably at approximately 90 degrees. The bone milling device may include a peripheral flange which serves as the guide.

### BRIEF DESCRIPTION OF THE DRAWINGS

FIGS. 1A and B shows a schematic view of a femur and tibia where A, sections of bone are being removed, and B, endoprostheses are being inserted, in a manner typical of the prior art.

FIG. 2A is a schematic illustration of the top view of a template and mill of the present invention.

FIG. 2B is a schematic illustration of a template and mill of the present invention viewed from the side at an angle.

FIG. 3 is a schematic illustration of a side view of a template and mill of the present invention.

FIGS. 4A-C shows a cross-sectional view of a groove receiving a drill flange.

FIGS. 5 A-C shows A, a perspective top view of a template with a pre-bent tab, B, a cross-sectional view of a mill head engaged with the template with a pre-bent tab, and C, perspective side views of various embodiments of pre-bent fastening means.

FIG. 6A-D is a schematic perspective view illustrating the use of a template and drill of the present invention.

FIG. 7A shows examples of tibial endoprostheses.

FIG. 7 B-D show perspective top views of templates of the invention.

FIG. 9 shows a schematic perspective representation of a femoral template.

FIG. 10 shows a perspective view of a femoral mill.

FIG. 11A-C shows A, perspective angled view of femoral mill and guide frame; B and C, side cross-sectional views of femoral mill and guide frame.

FIG. 12 shows a side cross sectional view of a femoral milling apparatus.

FIGS. 13A and B shows a top view of a guide frame.

### DETAILED DESCRIPTION OF THE PREFERRED EMBODIMENTS OF THE INVENTION

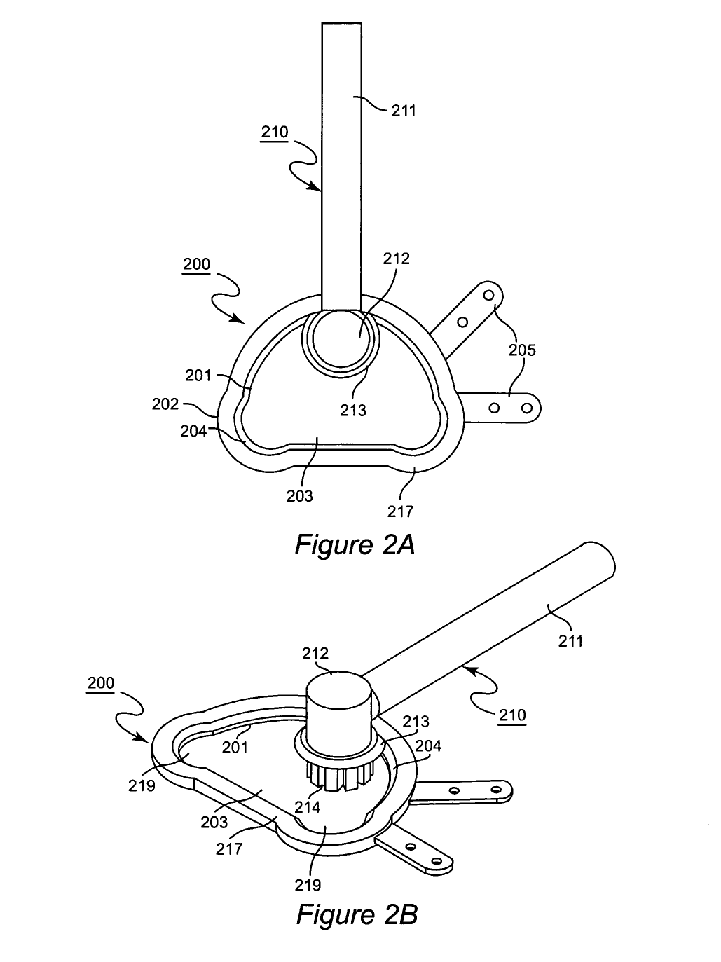

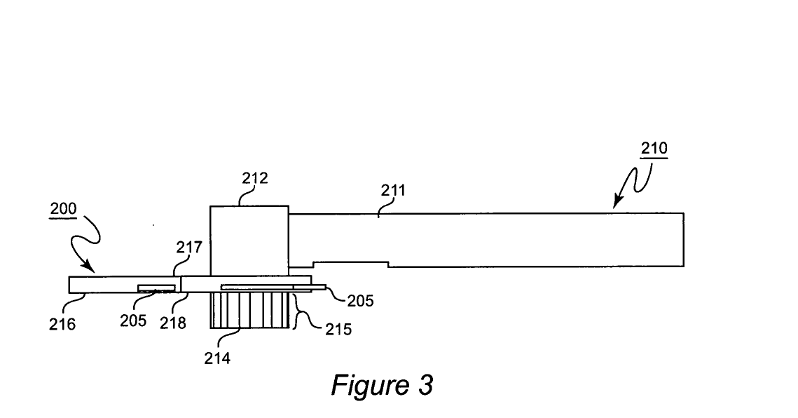

A top perspective view of a template and milling device of the present invention is shown in FIG. 2A. Referring to the Figure, template 200 is a frame with a top surface 217 and a bottom surface 216 (not shown; see FIG. 3). The frame of the template 200 is formed in a roughly hemispherical shape and bounded with an inner edge or rim (internal sidewall) 201 which defines an inner perimeter of the template, and an outer edge or rim (outer sidewall) 202 that defines the outer perimeter of the template. Inner rim 201 further defines a peripheral boundary of a central open space, central opening 203. Guide track or groove 204 runs along the entire length of the inner edge 201 of template 200. The frame is typically of a thickness of about 0.05 inches. Attachment means (e.g. attachment tabs 205) extend outward horizontally from template 200 and contain holes 206. In a preferred embodiment, attachment tabs 205 are bendable and may be scored to facilitate bending. The tabs are capable of being bent toward the bone, and a fastening means (e.g. a tack, pin, etc.) may be inserted through one or more of holes 205 in order to secure the template to the bone, e.g. to the tibia along the front of the tibia. Instead of or in addition to scoring tabs 205 to facilitate bending, the tabs may be made of material that is of a composition or thickness (or both) that makes the tab amenable to bending yet strong enough to offer robust support to the template when fastened to the bone. In a preferred embodiment, the thickness of the tabs is about 0.050 inches. Further, the precise shape and number of the fastening means need not be exactly as depicted in FIG. 2, but may be any useful shape or number, so long as the fastening means can be bent toward the bone, and contains holes through which a fastening means can be inserted into the bone. Examples of suitable shapes include but are not limited to hemispherical tabs, a single wider tab, rectangular shaped tabs, several (e.g. about 3 or more) narrower tabs, etc. Further, the tabs may be either capable of being bent during surgery, or may be pre-bent to an appropriate position prior to use.

Also depicted in FIG. 2 is tibial mill 210, which comprises a driving member 211, and a mill head 212 with flange (edge) 213 which extends outward along the circumference of the mill head 212. Driving member 211 extends away from template 200 and is contiguous with mill head 212 and thus can be used as a guide to direct the movement of mill head 212 in the guide track or groove 204 using the flange edge 213. Driving member 211 also connects the mill to a power source/driving means (not shown) or contains a power source/driving means.

As can be seen, with reference to FIG. 2B, flange 213 is of a size and shape that allows it to reversibly engage with guide track 204. Engagement of flange 213 with guide track 204 (i.e. insertion of flange 213 into guide track 204) causes mill head 212 to track along the inner perimeter of template 200 as mill 210 is moved, flange 213 riding along in guide track 204. As can be seen, this results in limiting the movement of the mill head 212 so that it is confined to space 203 within the template. Mill head 212 may be of any suitable shape but is preferably cylindrical.

FIG. 2B also shows a milling or cutting means (e.g. a burr) 214 attached to mill head 212 below flange 213. Burr 214 preferably has abrasive or cutting teeth on its distal end as well as along its sides, and may be, for example, an industrial end mill, router bit, or other suitable cutting means. As can be seen, mill head 212 and attached burr 214 extend downward from driving member 211 at approximately a 90° angle (e.g. a knee mill). When mill head 212 is positioned within template 200 and flange 213 is engaged with guide track 204, burr 214 extends through space 203 and, as shown in FIG. 3, down below the level of a bottom surface 216 of template 200. Cutting means 214 thus cuts into the bone to a depth limited by the distance that it extends beyond bottom surface 216 (distance 215 in FIG. 3). It should be understood that in FIG. 3, flange 213 is understood to be reversibly engaged with guide track 204 of the template, although neither is shown.

The result of this arrangement of template and mill is that when the template is affixed to a bone surface, the mill head and burr can be placed within the template and guided, via movement of the driving member 211, along the inner rim of the template. By engaging the flange 213 and the guide track 204, the motion of the burr can be restricted to lateral movement along the inner perimeter of the template. Further, the engagement also controls the depth of milling. That is, burr 214 is held at a constant vertical level by the engagement of the flange edge 213 in the guide track or groove 213, resulting in a uniform, level routing of the bone at a depth equal to the distance that the bottom surface of the cutting means extends beyond the bottom surface of the frame.

The depth of the cut may be determined by the relative placement and/or by the vertical length of burr 214. Referring to FIG. 3, vertical length 215 of burr 214 can be varied to achieve deeper or shallower cuts. Alternatively, the placement of flange 213 on the mill head can likewise be varied to raise or lower the position of the burr. Further, the thickness of the template itself and the positioning of the track or groove within the template may be altered so that engagement of the flange with the groove results in a higher or lower positioning of the burr, as desired.

With reference to FIG. 2B, bulbous regions 219 of the template allow the edge of burr 214 to extend over to a point which is coextensive with the peripheral corners of the insert to be positioned in the bone. In this way, relatively less bone needs to be removed in order to accommodate the insert.

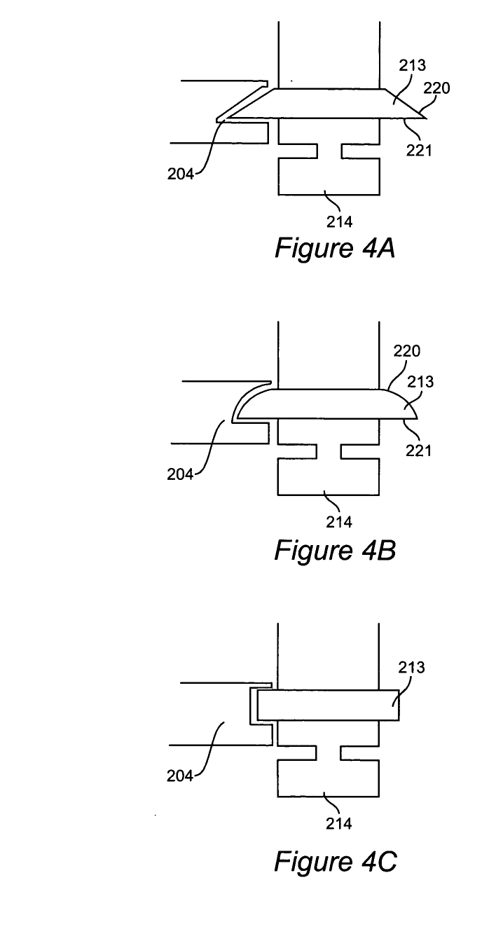

With reference to the connection between the mill head flange 213 and groove 204, “reversibly engaged” means that the edge fits into the groove in a manner that results in a stable but not permanent attachment. Some exemplary attachments are shown in FIG. 4A-C where the flange is depicted in the shape of an acute angle (A) with a flat top surface 220 and flat bottom surface 221, or (B) with a curved top surface 220 with a flat bottom surface 221, or C) a rectangular shape, and is received within the groove 204 which has a complementary shape, as shown. Those of skill in the art will recognize that many designs for coupling a flange and groove in this manner could be utilized. Preferably the bottom (underside) of the flange is flat and contacts a corresponding flat receiving surface in the groove. In general, in order to form a stable connection, the flange will extend into the groove in the range of about 0.1 mm to about 10 mm, and preferably about 1.0 mm to about 3.0 mm.

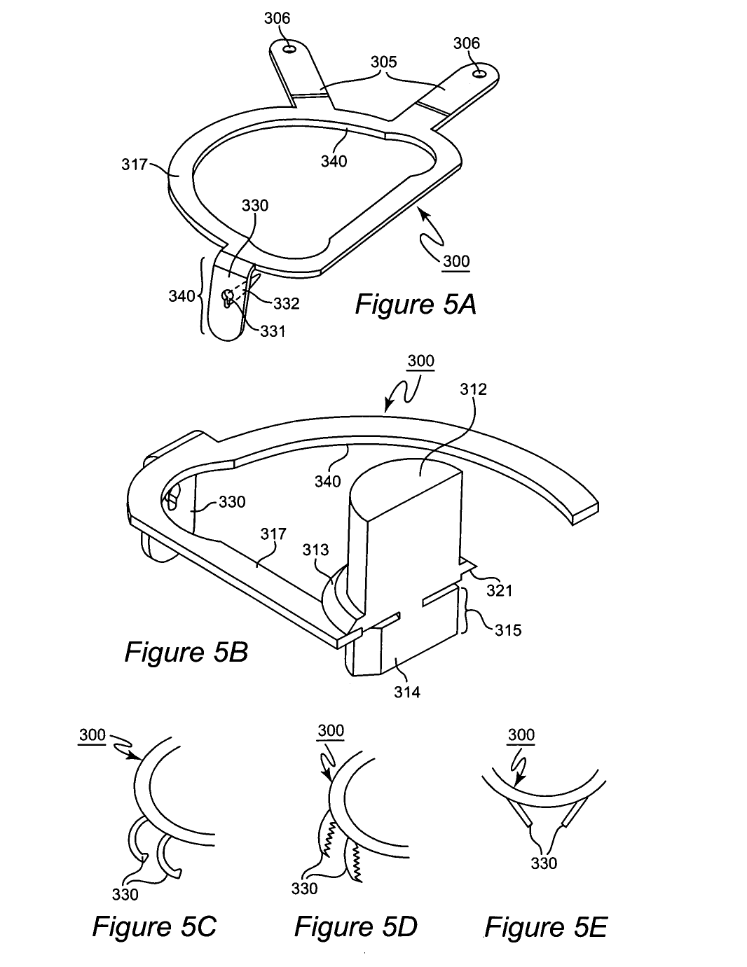

FIG. 5 A depicts a perspective view of yet another embodiment of a tibial template 300 of the present invention, and FIG. 5B depicts a cross-sectional view of a mill head 312 engaged with template 300. In this embodiment, the template does not contain a groove or guide track. Rather, the milling head engages the template with a simple “L” shaped geometry, i.e. flat underside 321 of flange 313 rests on flat upper surface 317 of template 300. Vertical inside edge 340 of template 300 guides the cutter within the template, delimiting the horizontal movement of the mill head, and flat, horizontal surface 317 supports the mill head and prevents the cutter from cutting too deeply into the bone. Cutting means 314 cuts into the bone only to a depth equal to the distance that it extends below the bottom surface of the template, distance 315 in FIG. 5.

The template embodiment pictured in FIGS. 5A and B also illustrates an added feature of a built-in pre-bent tab 330 on template 300. The purpose of pre-bent tab 330 is to provide a means for stabilizing placement of the template on a tibial surface that is to be resectioned. Because many vital nerves and blood vessels are located at the rear of the knee, during knee surgery, an incision is made only at the front of the knee in order to minimize the risk of damage to the nerves and blood vessels. Thus only the front of the knee bones are exposed, and securing of the template to the tibial surface can be done only from the front, the back of the knee being inaccessible to the surgeon. However, a pre-bent fastening means such as the pre-bent tab pictured in FIG. 5 offers a way to stabilize the template from the rear. When placing template 300 on the tibial surface that is to be resectioned, the template is first pushed back toward the back of the tibia in order to allow pre-bent tab 330 to drop over the back of the tibial surface. Template 330 is then pulled slightly forward to engage pre-bent tab 330 with the back of the knee, and additional fastening means (e.g. bendable tabs 306) are used to more fully secure the template at the front, e.g. with pins or tacks. In order to facilitate engagement of the pre-bent tab with the back of the knee, the inside surface of the tab (i.e. the surface that contacts the bone) may be made of or covered with material that reduces slippage of the tab when in contact with the bone, i.e. that provides traction for holding the tab in place. Alternatively, the inside surface of the tab may contain protrusions that roughen the surface (e.g. one or more small spikes, teeth, granular protuberances, other raised or relief surfaces) in order to promote reversible attachment of the pre-bent tab to the rear of the tibia during the milling process. FIG. 5A shows the use of a single tack 332 for holding the prebent tab 370. However, it should be understood that this tack could be eliminated, and the bone contacting surface could simply abut against the bone. After milling is complete, the template is removed by being pushed backward a sufficient distance to release the inside surface of the pre-bent tab from contact with the bone, and the template is then removed. This is accomplished by unbending tabs 306 at the front of the frame and using the tabs 306 to push the entire frame rearward enough to move the prebent tab 330 away from the bone. The space available to the surgeon during knee surgery is very limited. Thus, the depth 340 of the pre-bent tab is typically about 2 mm or less. The shape of the pre-bent fastening means need not be tabular as illustrated in FIGS. 5A and B, but may be more prong- or fish hook-shaped, and may be positioned at an angle, as illustrated in FIG. 5C-E. Further, more than one pre-bent fastening means may be located on the template. A pre-bent tab may be included in any of the templates described herein, e.g. those with a guide track, as shown in FIG. 2.

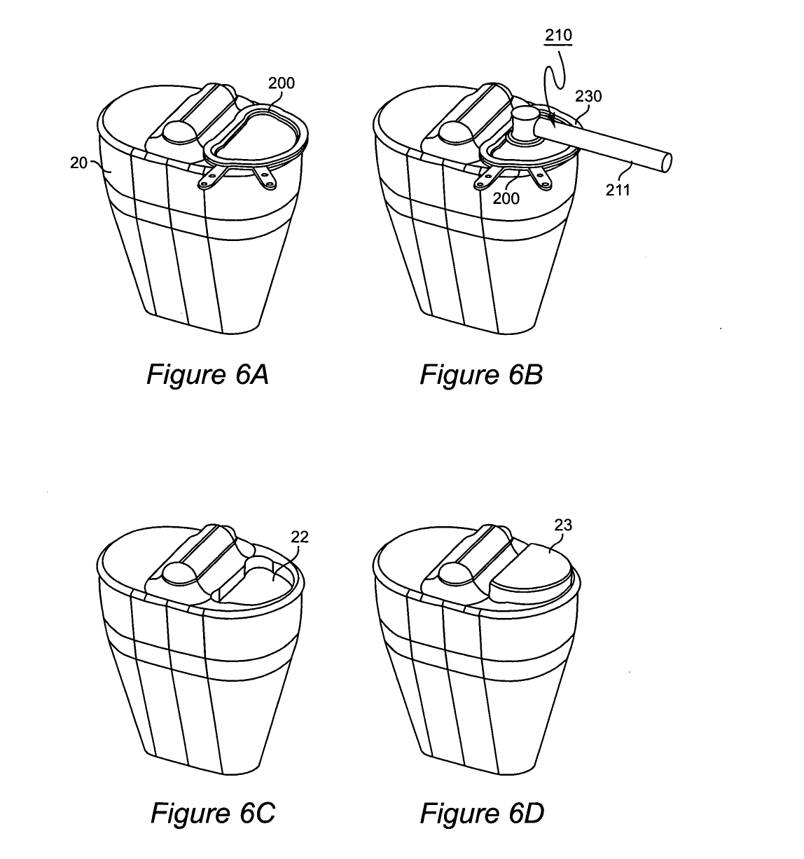

Use of the template and mill of the present invention to form a cavity for receiving a tibial prosthesis is illustrated in FIG. 6A-D. FIG. 6A shows a tibia with template 200 attached. FIG. 6B shows mill 210 engaged with the template 200 during milling of the bone. After attachment of the template to the bone, the surgeon positions the cutting means (e.g. burr) of the mill on the surface of the bone within the circumference of the template and mills, free hand, downward into the bone to a suitable depth, e.g. about 2 mm. The surgeon then mills laterally across the bone surface toward the interior wall of the template. Both vertical and horizontal milling is possible because the burrs that are used in the milling device of the present invention cut both vertically and horizontally. When the burr reaches the internal wall of the template, the flange of the milling device can be either engaged in the guide track or on top of the template (frame) and abuting the interior peripheral wall, and the surgeon then moves the milling device along the interior wall of the template with the mill flange engaged in the guide track, thereby restricting the lateral movement of the burr to within the area bounded by the template, and limiting the depth of the milling to that determined by the extension of the burr beyond the bottom of the template. The fit of the flange into the groove or the fit of the L-shaped region on the side of the mill, precludes the mill from tipping beyond a parallel position. This provides a cavity in the bone with a smooth, uniformly flat surface at least along the interior wall of the template, for stable placement of an implant. If, during initial freehand milling, the surgeon mills the center of the cavity slightly deeper than the surrounding area, this irregularity can easily be filled by applying bone cement. Likewise, if the initial freehand milling is of insufficient depth to match the surrounding area, the surgeon can freehand mill the center of the cavity again. It may also be possible to eliminate the free hand drilling by using a burr and template combination which allows all of the bone to be removed with one movement of the bone mill around the internal periphery of the template.

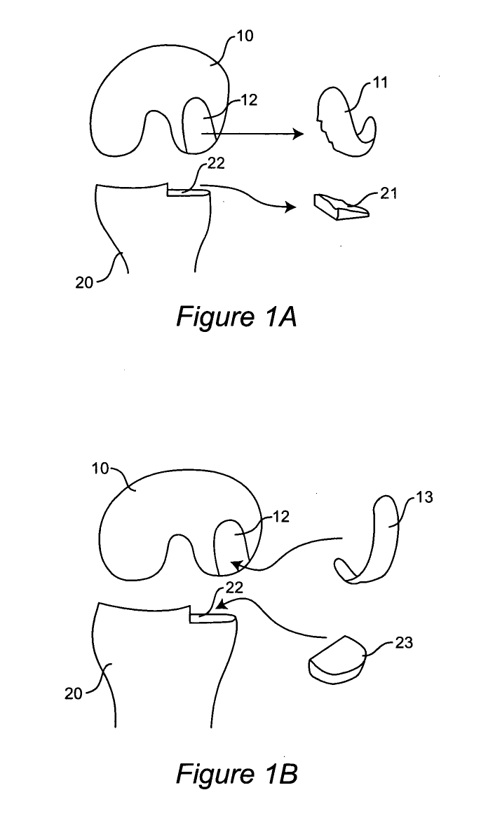

FIG. 6C shows a cavity 22 in the tibia made by the milling process of the present invention. As can be seen, in contrast to the prior art procedure illustrated in FIG. 1A, use of the template of the present invention produces a cavity in the bone that is completely surrounded by bone, i.e. is encompassed by a rim of bone 25, and thus less bone is removed during the milling process. As a result, the fit of the prosthesis within the cavity, as shown in FIG. 6D, is more precise and stable compared to a prosthesis placed on a typical L-shaped tibial cut (FIG. 1B). The rim of bone 25 serves to prevent misalignment of the prosthesis. Further, leaving more of the original bone is highly advantageous in the event that further knee operations are necessary, giving a surgeon more original bone surface to work with, e.g. during a complete knee replacement. In FIG. 6, a single milling procedure is illustrated. However, those of skill in the art will recognize that more than one milling may be undertaken. For example, a rough milling may first be carried out with a coarse burr to quickly remove most of the bone, followed by a second milling with a fine burr in order to smooth the surface and sides of cavity 22. Furthermore, while FIGS. 6A-D illustrate placement of an insert in the tibia, it will be recognized by those of skill in the art that the methods, templates, and kits of this invention can be practiced in other bones and with other prosthetic shapes.

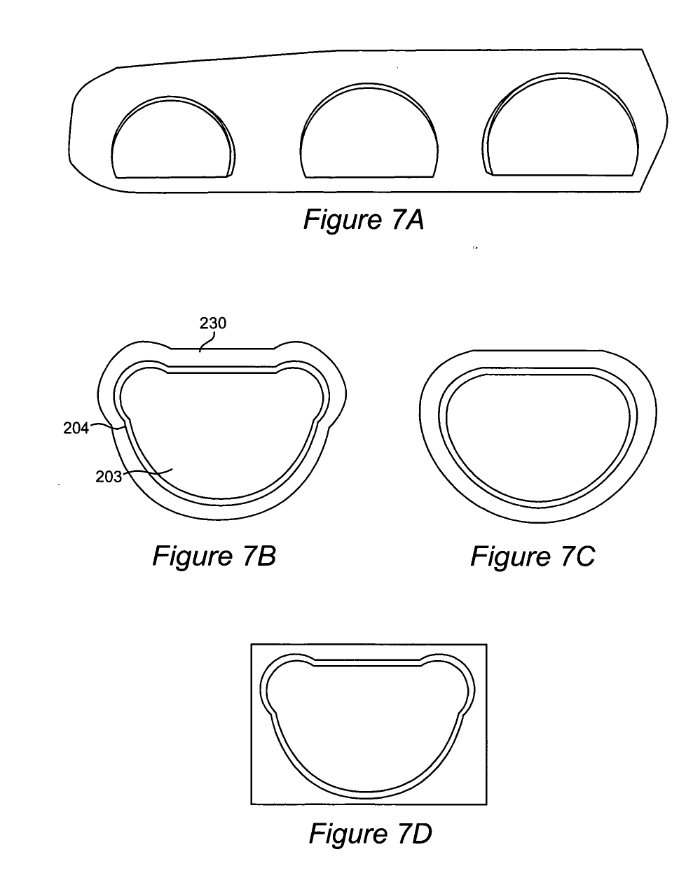

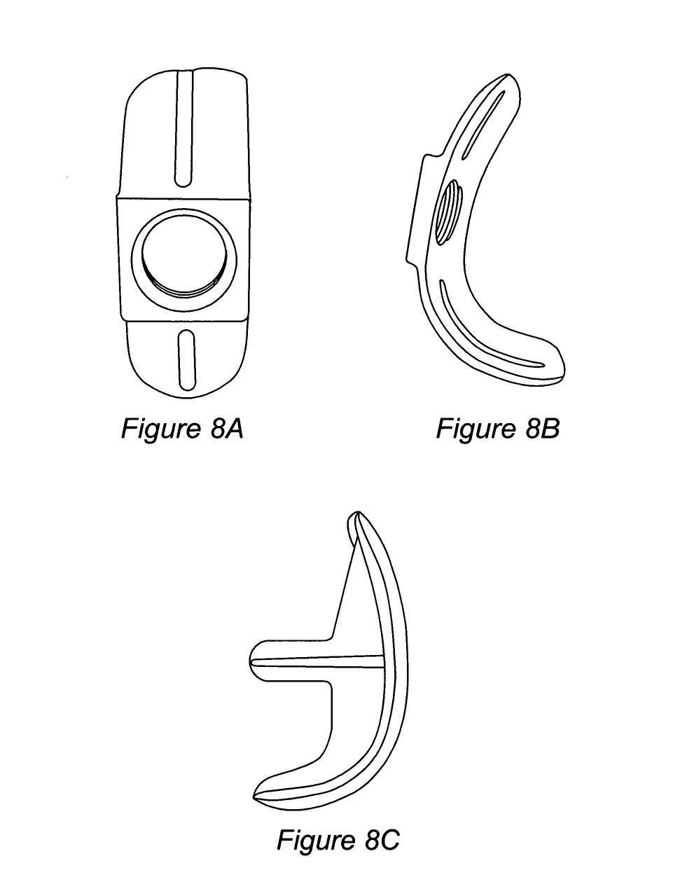

Tibial endoprostheses typically have a truncated circular shape and come in several standardized sizes as depicted in FIG. 7A. Likewise, the templates of the present invention are manufactured in several different sizes so that the central opening 203 accords with the size of the prosthesis to be utilized. Because the bottom cutting surface of a burr typically forms a circle and cannot cut angles, the shape of central opening 203 is designed without angles, and the milled cavity that is formed in the bone extends beyond the angular edges of the prosthesis. Examples of exemplary designs as shown in FIGS. 7B and C. As can be seen, the inner rim of the template is curved to accommodate the circular burr. The space in the bone which is formed by the use of the mill and template of the present invention thus has rounded edges instead of angles as in the prosthesis, resulting in a small amount of space between the edge of the prosthesis and the bone. This intervening space may be filled with a suitable substance such as bone cement in order to stabilize the implant within the cavity.

The space available to the surgeon while performing knee surgery is very limited. Thus, the overall size of the template should be as small as practically possible. In a preferred embodiment, and as depicted in FIG. 7B, the template surface 230 surrounding the central opening 203 is wide enough to contain the groove 204 (if a groove is employed) but is otherwise as narrow as possible, preferably in the range of from about 0.1 to about 10 mm, and preferably from about 2 mm to about 4 mm.

While it is preferred to keep the width of template surface 230 as narrow as possible, the shape of the surface need not be limited to that depicted in FIG. 7B. It is the central opening 203 that dictates the dimensions of the bone that will be removed, and that should be fashioned with a design approximating that of the prosthesis. The outer rim of the template may be of any practical shape (such as a rectangle or other polygon) as is depicted in FIG. 7D. An alternative embodiment of the template is also shown in FIG. 7C.

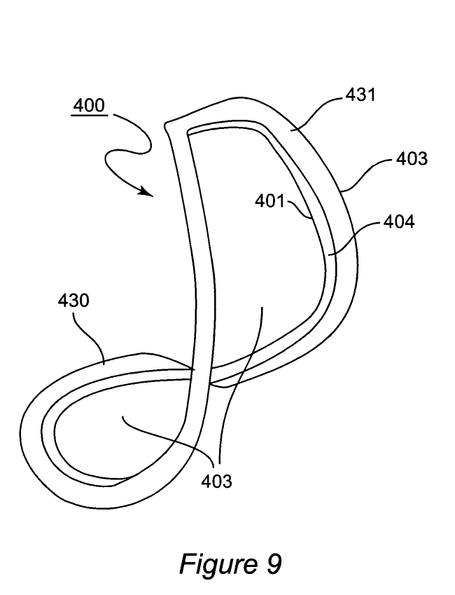

In addition to providing a template and drill for tibial endoprostheses, the present invention also provides templates and drills for femoral endoprostheses. FIGS. 8A and B show front and side perspective views, respectively, of one typical femoral prosthesis designed with an opening for a pin for attachment to the femur, and FIG. 8C shows a perspective side view of another typical femoral prosthesis with a built-in pin for attachment to the femur. As illustrated in FIG. 1B, where 13 represents the prosthesis, prosthetic devices designed for the femur are curved in order to fit the curvature of the bone. Thus, the femoral templates of the present invention are also curved in order to fit the contours of the femur. A schematic representation of a template for a femoral implant is shown in FIG. 9, which shows a template 400 with top surface 430, bottom surface 431, inner edge or rim (sidewall) 401, and groove or guide track 404 running along internal sidewall 401 of template 400. As is the case for the tibial implant, means of attachment (bendable or pre-bent) may also be provided but are not shown in FIG. 9. In use, the template is fixed securely to the femur and a knee drill having the attributes discussed above for the tibial template is engaged with an edge reversibly inserted into the guide track 404. Alternatively, the invention also contemplates a femoral template without a guide track (analogous to the tibial template of FIG. 5). As is the case for the tibial templates of the present invention, the femoral guide track 404 serves as a guide to limit the lateral movement of the drill, and the depth of milling is controlled by the vertical placement of the burr with respect to the bottom surface of the template, i.e. the distance of extension of the burr beyond the bottom surface of the template.

In order to use a template of the present invention, a trained professional such as an orthopedic surgeon chooses a template of suitable size for use in a particular operation. Tibial templates will typically be provided in a variety of sizes, e.g. small, medium and large. Further, for each size category, four sub-categories of templates (left and right medial, and left and right lateral) will be available. For femoral templates, left and right medial, and left and right lateral will typically be provided. After selection of the appropriate template, the template is secured to the appropriate bone surface (tibial or femoral) by K-wire or bone tack.

The templates of the present invention may be made from a variety of suitable materials, including but not limited to plastics and other synthetic polymers, metals, ceramics, or combinations of there materials. In a preferred embodiment, the material is stainless steel metal.

Kits for performing surgery may include a disposable or reusable bone mill and one or more template frames. Preferably, a plurality of frames of different sizes will be included. As discussed in conjunction with FIGS. 5A and 5B, the disposable bone mill would have a specific region (e.g. L-shaped) which mates with the frame. Alternatively, as discussed in FIGS. 2, 4 and 9, the bone mill may be configured to ride in a guide track of the frame. One may also include one or more implants (e.g. tibia implants of different sizes in combination with one or more femoral implants).

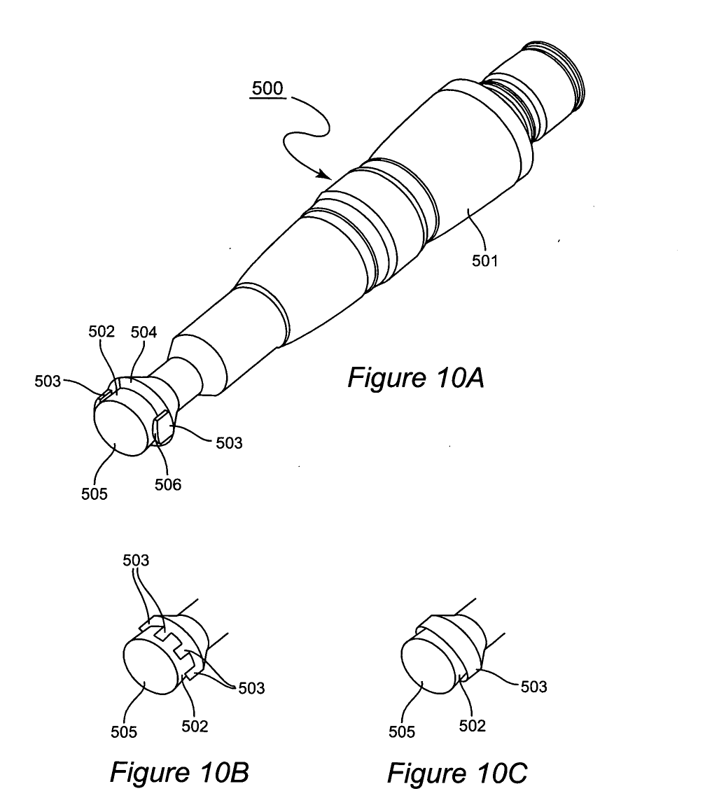

The present invention further provides a bone mill with a design that is especially useful for milling the femur for placement of a femoral prosthesis. The femoral bone mill does not require the use of a template. The femoral bone mill 500, illustrated in FIG. 10, comprises drive member 501 which connects to or houses a power source and serves as a handle, and a cutting means 502 disposed at one end of drive member 501. The bone mill further comprises supports 503 protruding from surface 504 of drive member 501, and along the sides of cutting member 502, i.e. the support means is a radial support means that radially surrounds an upper portion of cutting means 503. Support means 503 extend down the sides of cutting member 502 to cover all but preferably about 2 mm of a bottom portion of cutting member 502. Thus, when femoral mill 500 is positioned with bottom surface 505 of cutting means 503 in contact with a bone surface, and power is supplied to the mill, cutting means 503 mills the bone directly under the lower surface 505 of the mill but only up to a depth of 2 mm, i.e. up until the supports come into contact with the bone surface. Then, by directing the path of the mill over the surface of the bone, a groove 2 mm deep with a width equal to the diameter of the cutting means, (e.g. about 5 mm) can be milled into the bone surface. A lower edge 506 of support 503 rides on the surface of the bone and prevents the depth of the cut from exceeding 2 mm. Those of skill in the art will recognize that, while the drive member 501 has been depicted as straight and elongated in FIG. 10, this need not be the case. The drive member may be of any suitable shape (e.g. curved, placed at a 90° angle from the mill head, etc.), so long as the position of the mill head can be directly controlled by movement of the drive member. Support means 503 may only partly circumscribe the circumference of cutting means 502 as shown in FIG. 10, where support means 503 has the appearance of ears extending downward from surface 504 of the drive member. While only two supporting ears are depicted in FIG. 10, those of skill in the art will recognize that support means may be utilized, the support means then having the appearance of teeth surrounding the upper portion of the cutting means as illustrated in FIG. 10B. Alternatively, support means 503 may completely circumscribe the circumference of the upper portion of cutting means 502, the rim of the 503 riding on the bone during milling, as illustrated in FIG. 10C. While this type of bone mill is especially suitable for milling the femur, those of skill in the art will recognize that its use need not be limited to the femur. Rather, the mill may be utilized for milling of any suitable bone surface. The femoral bone mill may be a one-use (disposable) mill.

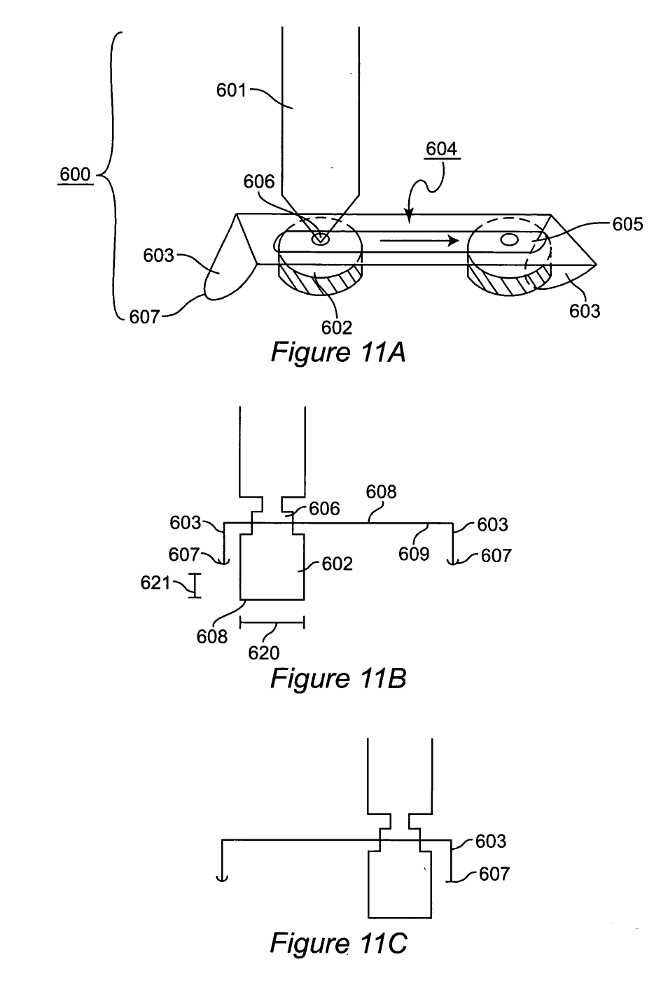

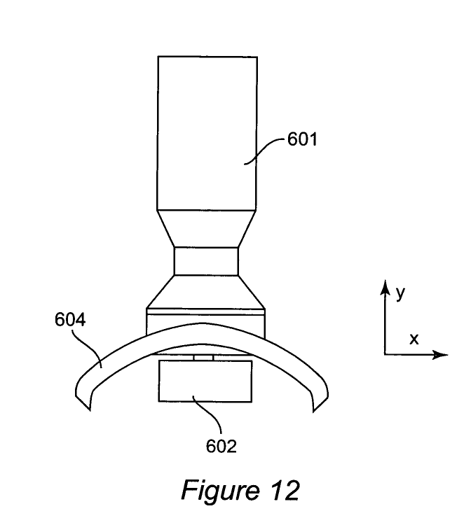

The present invention also provides a bone milling apparatus as schematically illustrated in FIG. 11. The apparatus comprises a cutting device 600 and a frame 604. The cutting device 600 comprises a drive member 601 which houses (or connects to) a power source, a cutting means 602 disposed at an end of the drive member, and a chucking mechanism 606 that connects drive member 601 to cutting means 602. Frame 604 has a top surface 608 and a bottom surface 609, and a slot 605 extending through the frame. Frame 604 further comprises at least one support means 603, which is disposed from bottom surface 609 of the frame. Chucking means 606 of cutting device 600 extends through slot 605 as illustrated. In the apparatus, the bottom surface 608 of cutting means 602 projects beyond a lower extremity 607 of support means 603 by a distance equal to a depth of a cut made by the cutting means. In other words, the depth of the cut that can be made by cutting means 602 is limited by how far beyond lower extremity 607 of support means 603 the lower surface 608 of the cutting means extends, the distance being represented by distance 621 in FIG. 11B. In FIG. 11, support means 603 are depicted as tabular legs extending downward from both ends of a rectangular frame. However, those of skill in the art will recognize that the precise shape and disposition of support means 603 may vary, and may include, for example, curved tabular legs, cylindrical pillars (e.g. one disposed at each apex of a rectangular frame), etc. Further, multiple support means may be disposed along the edges of the frame, or close to the edges of the frame. The frame itself need not be rectangular but may be of any suitable shape (e.g. an oval) that allows placement of a slot for positioning of the cutting device, and disposition of support means to limit the depth of the cut made by the cutting means. Further, the surface of the frame need not be flat, but may, for example, be curved, for example, as shown in FIG. 12. FIG. 12 is a side cross sectional view of a femoral bone milling apparatus in which the frame is curved, and the slot (which cannot be seen in FIG. 12) runs along the frame in the x direction, according to the x-y plane indicated in FIG. 12.

With reference to FIG. 11, in order to perform a cut with this apparatus, the cutting device is preferably locked into a first position in the frame as shown in FIG. 11B by a lock or latch mechanism (not shown), the entire device is positioned at a suitable initial position on the bone (typically pre-marked), and a cut is made downward into the surface of a bone directly beneath cutting means 602, until the tips 607 of supports 603 rest on the surface of the bone. The apparatus is then moved along the bone by the operator (e.g. a surgeon) using the driving member 601 as a handle, support extremities 607 riding on the surface of the bone. The operator controls the length of the channel that is milled in the bone, which will typically (although not necessarily) be milled in a straight line between positions on the bone surface that were premarked prior to milling. The width of the first groove that is cut will equal the diameter 620 of cutting means 602 (e.g. about 5 mm to about 10 mm), and cutting means 602 will cut into the bone surface only to a depth 621 which is the distance from the bottom edge 607 of support 603 to bottom surface 608 of cutting means 602, typically about 2 mm. In order to make a wider channel of the same depth in the bone (e.g. a channel that is 20 mm wide and 2 mm deep if a 10 mm burr is used), after milling the first channel, the cutting device 600 is released from the first position in the frame and moved via drive member 601 along slot 605 to a second position in the frame, shown in FIG. 11C. The cutting device is then locked into the second position. A second groove immediately adjacent to or slightly overlapping the first groove is then milled into the bone surface by pulling the entire device along the bone and back to the initial position, resulting in a single groove with a width of twice the diameter of the burr (e.g. about 20 mm if a 10 mm burr is used) and a depth of 2 mm. Alternatively, to cut the second groove, the device may be placed at the initial position and the second groove may be milled adjacent to the first and in the same direction that the first groove was milled. While milling the second groove, supports 603 rest on and ride or slide over the bone surface, straddling the first groove and the incipient second groove.

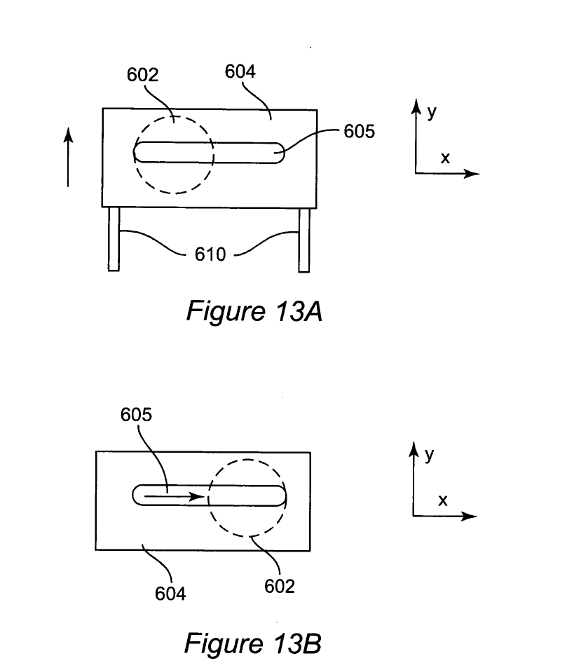

In one embodiment, supports 603 rest on the surface of the bone (or on the cartilage or other tissue the covers the bone) and slide along the surface during milling. In another embodiment of the invention, bottom edges 607 of supports 603 are pointed or sharpened and as the device is drawn over the surface, a track is cut into the cartilage by the sharpened edges. As a result, when additional grooves are milled adjacent to the first groove, supports 603 of the device will run along the carved track, and alignment of the device in the x direction of an x-y plane is thereby maintained. For example, see FIG. 13 A and B, which show a top view of changing the cutting device from a first position (A) to a second position (B) in an x direction of an x-y plane. In FIGS. 13A and B, milling of channels in the bone is done in the y direction of the x-y plane, and may be guided by tracks 610 carved into the surface during the first pass of the device.

While a frame with two positions is shown, those of skill in the art will recognize that the slot may designed with three or more positions for receiving the cutting device. The positions may overlap to varying degrees. In addition, the positions may be at discrete, fixed locations along the slot, or alternatively, the position of the cutting device in the slot may be infinitely adjustable along the length of the slot (i.e. the cutting device may be locked into position at any location along the slot). Thus, the method is not restricted to milling two adjacent channels in a bone. Rather, several channels may be milled. For example, a relatively narrow burr (5 mm) may be used to mill a 20 mm channel by traversing the bone four times, instead of using a relatively wide burr (10 mm) to mill a 20 mm channel in two passes of the device. Making several passes with a narrower burr may provide more definition (e.g. curvature) to the final channel than is achieved with fewer passes with a wider burr.

In yet another embodiment of the invention, milling of the bone is done in the direction of the slot, i.e. the mill in not locked into a position but slides along the slot, the slot acting as a guide for the mill.

Those of skill in the art will recognize that, with respect to the drills (e.g. the 90° knee drill) used in some embodiments of the present invention, many such drills are known and may be employed in combination with many known suitable burrs, depending on the needs and preferences of the surgeon and the available resources. Examples of suitable drills and burrs include but are not limited to various orthopedic and dental drills. In a preferred embodiment, the drill and burr combination is a 90° drill with a total height not exceeding about 15 mm. Likewise, for the femoral mill, many suitable burrs for use as a cutting means are known and may be employed. Further, the precise attachment of the burr to the neck and drive member (e.g. the angle of attachment, the length of the handle, etc.) may vary.

In a preferred embodiment of the present invention, the templates, mills and apparatuses of the present invention are used for milling bone during unicompartmental knee replacement, and function to guide milling of the tibia and/or femur. However, those of skill in the art will recognize that the invention is not limited to templates for this purpose. The templates of the present invention may be used for milling any bone. In other embodiments, the size and shape of the template may be designed to fit the bone surface that is to be milled, and the depth of the cut may be adjusted as necessary. Examples of other types of bone milling that can be facilitated by the templates and methods of the present invention include but are not limited to milling for complete knee replacement, for the placement of prosthetic or artificial bone during reconstructive or plastic surgery, total hip replacement, arthroscopic knee surgery, etc.

The present invention further provides kits containing materials for milling bone. In preferred embodiments, the materials are for tibial bone milling, femoral bone milling, or both in a single kit. A kit may include at least one template and, preferably, at least one one-use, disposal bone mill. The template may be a tibial and/or femoral template, and may be designed with or without a guide track. A mill such as a 90° knee mill, and/or a femoral mill with built-in supports, and/or a femoral mill with a guide frame as described herein, or a combination of these, may be included in the kits of the present invention. The kit may include multiple prosthetic implants of differing sizes and templates of differing sizes. A kit may include both a 90° knee mill and a femoral mill as shown in FIG. 10 or FIG. 2C, as well as one or more tibial frames.

While the invention has been described in terms of its preferred embodiments, those skilled in the art will recognize that the invention can be practiced with modification within the spirit and scope of the appended claims. Accordingly, the present invention should not be limited to the embodiments as described above, but should further include all modifications and equivalents thereof within the spirit and scope of the description provided herein.

We claim:

1. A template for bone milling, comprising:
a frame having a top, a bottom, one or more external sidewalls, and one or more internal sidewalls, said frame having one or more openings extending there through from said top to said bottom wherein at least one of said one or more internal side walls defines a peripheral boundary of each of said one or more openings;
a guide track formed in said one or more internal sidewalls, said guide track receiving a guide of a bone milling device whereby said bone milling device may be moved about said peripheral boundary using said guide track.

2. The template of claim 1 further comprising a means for removably securing said frame to a bone which is to be milled.

3. The template of claim 2 wherein said means for removably securing includes one or more tabs projecting from said frame which have one or more securing points which may be secured to a bone.

4. The template of claim 3 wherein said one or more tabs project from said one or more external sidewalls.

5. The template of claim 1 wherein said guide track is positioned approximately midway between said top and said bottom of said frame.

6. The template of claim 1 wherein said guide track has a flat lower surface which is approximately parallel to said top and said bottom of said frame.

7. The template of claim 1 wherein said guide track has an angled upper surface which projects at an angle from said lower surface to a point relatively closer to said top of said frame than said bottom of said frame.

8. The template of claim 1 wherein said guide track has an arcuate upper surface which extends from said lower surface to a point relatively closer to said top of said frame than said bottom of said frame.

9. The template of claim 1 wherein guide track has an arcuate lower surface and an angled upper surface which projects at an angle from said lower surface to a point relatively closer to said top of said frame than said bottom of said frame.

10. The template of claim 1 wherein said frame is curved to match one or more curves of a bone.

11. The template of claim 10 wherein said frame has a peripheral boundary in a shape configured to accommodate a femoral implant.

12. The template of claim 1 wherein said frame has a peripheral boundary in a shape configured to accommodate a tibial implant.

13. The template of claim 1 wherein said peripheral boundary has one or more bulbous regions.

14. A kit for partial knee replacement surgery; comprising:
a plurality of tibial frames, each of said tibial frames having a top, a bottom, an external sidewall, and an internal sidewall, each of said tibial frames having an opening extending therethrough from said top to said bottom of said tibial frame wherein said internal side wall defines a peripheral boundary of said opening, each of said plurality of tibial frames having an opening sized to match one of said plurality of tibial implants;
a guide track formed in said internal sidewall of each of said tibial frames, said guide track receiving a guide of a bone milling device whereby said bone milling device may be moved about said peripheral boundary using said guide track.

15. The kit of claim 14, further comprising at least one femoral frame having a top, a bottom, an external sidewall, and an internal sidewall, said at least one femoral frame having an opening extending therethrough from said top to said bottom of said femoral frame wherein said internal side wall defines a peripheral boundary of said opening wherein said opening is sized to match said at least one femoral implant.

16. The kit of claim 15 wherein said tibial frames are constructed from metal.

17. The kit of claim 14 wherein said tibial frames are constructed from plastic.

18. The kit of claim 14 wherein said tibial frames are constructed from ceramics.

19. The kit of claim 15 wherein said femoral frames is constructed from metal.

20. The kit of claim 15 wherein said femoral frame is constructed from plastic.

21. The kit of claim 15 wherein said femoral frame is constructed from ceramic.

22. The kit of claim 14 further comprising means for removably securing each of said tibial frames to a tibia bone.

23. The kit of claim 22 wherein said means for removably securing includes one or more tabs projecting from said tibial frame which have one or more securing points which may be secured to a tibia bone.

24. The kit of claim 23 wherein said one or more tabs project from said external side wall of said tibia frame.

25. The kit of claim 15 further comprising means for removably securing said at least one femoral frame to a femur bone.

26. The kit of claim 14 wherein said guide track in each of said tibial frames is positioned approximately midway between said top and said bottom of said tibial frame.

27. The kit of claim 14 wherein said guide track in each of said tibial frames has a flat lower surface which is approximately parallel to said top and said bottom of said tibial frame.

28. The kit of claim 14 wherein said guide track in each of said tibial frames has an angled upper surface which projects at an angle from said lower surface to a point relatively closer to said top of said frame than said bottom of said tibial frame.

29. The kit of claim 14 wherein said guide track in each of said tibial frames has an arcuate upper surface which extends from said lower surface to a point relatively closer to said top of said tibial frame than said bottom of said tibial frame.

30. The kit of claim 14 wherein guide track in each of said tibial frames has an arcuate lower surface and an angled upper surface which projects at an angle from said lower surface to a point relatively closer to said top of said tibial frame than said bottom of said tibial frame.

31. The kit of claim 14 wherein said peripheral boundary of at least one of said plurality of tibial frames has one or more bulbous regions

32. The kit of claim 15 further comprising a guide track formed in said internal sidewall of said at least one femoral frame, said guide track receiving a guide of a bone milling device whereby said bone milling device may be moved about said peripheral boundary using said guide track.

33. The kit of claim 32 wherein said guide track in said at least one femoral frame is positioned approximately midway between said top and said bottom of said femoral frame.

34. The kit of claim 32 wherein said guide track in said at least one femoral has a flat lower surface which is approximately parallel to said top and said bottom of said femoral frame.

35. The kit of claim 32 wherein said guide track in said at least one femoral frame has an angled upper surface which projects at an angle from said lower surface to a point relatively closer to said top of said femoral frame than said bottom of said femoral frame.

36. The kit of claim 32 wherein said guide track in said at least one femoral frame has an arcuate upper surface which extends from said lower surface to a point relatively closer to said top of said femoral frame than said bottom of said femoral frame.

37. The kit of claim 32 wherein guide track in said at least one femoral frame has an arcuate lower surface and an angled upper surface which projects at an angle from said lower surface to a point relatively closer to said top of said femoral frame than said bottom of said femoral frame.

38. The kit of claim 14 further comprising a bone milling device.

39. The kit of claim 38 wherein said bone milling device is a one time use disposable.

40. The kit of claim 38 wherein said bone milling device has a milling bit which is angled from a drive member.

41. The kit of claim 40 wherein said milling bit is angled at approximately 90 degrees from said drive member.

42. The kit of claim 38 wherein said bone milling device includes a peripheral flange which serves as said guide.

43. A kit for orthopedic surgery; comprising:
a plurality of implants;
a plurality of frames, each of said frames having a top, a bottom, an external sidewall, and an internal sidewall, each of said frames having an opening extending therethrough from said top to said bottom of said frame wherein said internal side wall defines a peripheral boundary of said opening, each of said plurality of frames having an opening sized to match one of said plurality of tibial implants; and
a guide track formed in said internal sidewall of each of said frames, said guide track receiving a guide of a bone milling device whereby said bone milling device may be moved about said peripheral boundary using said guide track.

44. The kit of claim 43 wherein said frames are constructed from metal.

45. The kit of claim 43 wherein said frames are constructed from plastic.

46. The kit of claim 43 wherein said frames are constructed from ceramics.

47. The kit of claim 43 further comprising means for removably securing each of said frames to a bone.

48. The kit of claim 47 wherein said means for removably securing includes one or more tabs projecting from said tibial frame which have one or more securing points which may be secured to a bone.

49. The kit of claim 48 wherein said one or more tabs project from said external side wall of said frame.

50. The kit of claim 43 wherein said guide track in each of said frames is positioned approximately midway between said top and said bottom of said frame.

51. The kit of claim 43 wherein said guide track in each of said frames has a flat lower surface which is approximately parallel to said top and said bottom of said frame.

52. The kit of claim 43 wherein said guide track in each of said frames has an angled upper surface which projects at an angle from said lower surface to a point relatively closer to said top of said frame than said bottom of said frame.

53. The kit of claim 43 wherein said guide track in each of said frames has an arcuate upper surface which extends from said lower surface to a point relatively closer to said top of said frame than said bottom of said frame.

54. The kit of claim 43 wherein guide track in each of said frames has an arcuate lower surface and an angled upper surface which projects at an angle from said lower surface to a point relatively closer to said top of said frame than said bottom of said frame.

55. The kit of claim 43 wherein said peripheral boundary of at least one of said plurality of frames has one or more bulbous regions.

56. The kit of claim 43 further comprising a bone milling device.

57. The kit of claim 56 wherein said bone milling device is a one time use disposable.

58. The kit of claim 56 wherein said bone milling device has a milling bit which is angled from a drive member.

59. The kit of claim 58 wherein said milling bit is angled at approximately 90 degrees from said drive member.

60. The kit of claim 56 wherein said bone milling device includes a peripheral flange which serves as said guide.

61. A bone milling method, comprising the steps of:
affixing to a joint region of a bone a template having
(i) a frame having a top, a bottom, one or more external sidewalls, and one or more internal sidewalls, said frame having one or more openings extending therethrough from said top to said bottom wherein at least one of said one or more internal side walls defines peripheral boundary of each of said one or more openings, and
(ii) a guide track formed in said one or more internal sidewalls; and
milling a portion of said joint region of said bone using a bone milling device having a guide which is received in said guide track by moving said bone milling device about said peripheral boundary using said guide track to guide said bone milling device.

62. The method of claim 61 wherein said milling step leaves a central plateau in said joint region which is removed by said bone milling device without using said template.

63. The method of claim 61 further comprising the step of controlling a depth of milling by said bone milling device.

64. The method of claim 63 wherein said step of controlling is achieved by selecting a thickness of said frame affixed to said bone in said affixing step.

65. The method of claim 63 wherein said step of controlling is achieved by selecting a position of said guide track in said frame affixed to said bone in said affixing step.

66. The method of claim 63 wherein said step of controlling is achieved by selecting a size of a burr used in said milling step.

67. A template for bone milling, comprising:
a frame having a top, a bottom, one or more external sidewalls, and one or more internal sidewalls, said frame having one or more openings extending there through from said top to said bottom wherein at least one of said one or more internal side walls defines a peripheral boundary of each of said one or more openings; and
a pre-bent fastening means for removably securing said frame to a posterior surface of a bone which is to be milled.

68. The template of claim 67, wherein said pre-bent fastening means is at least one pre-bent tab.

69. The template of claim 67, wherein said pre-bent fastening means is at least one pre-bent hook.

70. The template of claim 67 further comprising a means for removably securing said frame to an anterior surface of a bone which is to be milled.

71. The template of claim 70 wherein said means for removably securing includes one or more tabs projecting from said frame which have one or more securing points which may be secured to a bone.

72. The template of claim 71 wherein said one or more tabs project from said one or more external sidewalls.

73. The template of claim 67 wherein said frame is curved to match one or more curves of a bone.

74. The template of claim 67 wherein said frame has a peripheral boundary in a shape configured to accommodate a femoral implant.

75. The template of claim 67 wherein said frame has a peripheral boundary in a shape configured to accommodate a tibial implant.

76. The template of claim 67 wherein said peripheral boundary has one or more bulbous regions.

77. The template of claim 67 wherein said template further comprises
a guide track formed in said one or more internal sidewalls, said guide track receiving a guide of a bone milling device whereby said bone milling device may be moved about said peripheral boundary using said guide track.

78. A bone milling method, comprising the steps of:
affixing to a joint region of a bone a template having
(i) a frame having a top, a bottom, one or more external sidewalls, and one or more internal sidewalls, said frame having one or more openings extending therethrough from said top to said bottom wherein at least one of said one or more internal side walls defines peripheral boundary of each of said one or more openings, and
(ii) a pre-bent fastening means for removably securing said frame to a posterior surface of a bone which is to be milled; and
milling a portion of said joint region of said bone using a bone milling device.

79. The bone milling method of claim 78, wherein said bone milling device has a flange with a flat surface that rests on said top of said frame, said bone milling device moving about said peripheral boundary using said internal sidewall to guide said bone milling device.

80. The bone milling method of claim 78, wherein said frame further comprises
a guide track formed in said one or more internal sidewalls, said guide track receiving a guide of a bone milling device whereby said bone milling device may be moved about said peripheral boundary using said guide track.

81. The method of claim 78, wherein said fastening means is at least one pre-bent tab.

82. The method of claim 78, wherein said fastening means is at least one pre-bent hook.

83. The method of claim 78 further comprising the step of controlling a depth of milling by said bone milling device.

84. The method of claim 83 wherein said step of controlling is achieved by selecting a thickness of said frame affixed to said bone in said affixing step.

85. The method of claim 83 wherein said step of controlling is achieved by selecting a size of a burr used in said milling step.

86. A bone milling kit, comprising:
a plurality of implants; and
a plurality of frames, each of said frames having a top, a bottom, an external sidewall, and an internal sidewall, each of said frames having an opening extending therethrough from said top to said bottom of said frame wherein said internal side wall defines a peripheral boundary of said opening, each of said plurality of frames having an opening sized to match one of said plurality of tibial implants, each frame comprising a pre-bent fastening means for removably securing said frame to a non-exposed surface of a bone which is to be milled.

87. The kit of claim 86 wherein said frames are constructed from a material selected from the group consisting of metal, plastic, and ceramics.

88. The kit of claim 86 further comprising means for removably securing each of said frames to an exposed surface of a bone.

89. The kit of claim 88 wherein said means for removably securing includes one or more bendable tabs projecting from said frame which have one or more securing points which may be secured to an exposed surface of a bone.

90. The kit of claim 89 wherein said one or more bendable tabs project from said external side wall of said frame.

91. The kit of claim 86 wherein said peripheral boundary of at least one of said plurality of frames has one or more bulbous regions.

92. The kit of claim 86 further comprising a bone milling device.

93. The kit of claim 92 wherein said bone milling device includes a cutting means with a peripheral flange with a bottom surface which rests on said top of said frame during milling, thereby controlling the depth of cutting of said cutting means.

94. The kit of claim 92 wherein said bone milling device is a one time use disposable.

95. The kit of claim 92 wherein said bone milling device has a milling bit which is angled from a drive member.

96. The kit of claim 95 wherein said milling bit is angled at approximately 90 degrees from said drive member.

97. The kit of claim 86 wherein said implants are tibial implants and said frames are tibial frames.

98. The kit of claim 86 wherein said implants are femoral implants and said frames are femoral frames.

99. A bone milling kit, comprising,
at least one frame having a top, a bottom, an external sidewall, and an internal sidewall, said at least one frame having an opening extending therethrough from said top to said bottom of said frame wherein said internal side wall defines a peripheral boundary of said opening; and
a milling device with a cutting means and a flange, a bottom surface of said flange riding on said top of said frame and a side surface of said flange abutting against said frame during milling, thereby controlling the depth of cutting by said cutting means.

100. The kit of claim 99 further comprising one or more means for removably securing said frame to a bone which is to be milled.

101. The kit of claim 100 wherein said one or more means for removably securing said frame to a bone is a pre-bent fastening means for removably securing said frame to an un-exposed surface of a bone which is to be milled.

102. The kit of claim 100 wherein said one or more means for removably securing includes one or more bendable tabs projecting from said frame which have one or more securing points which may be secured to an exposed surface of a bone.

103. The kit of claim 100 wherein said at least one frame is a tibial frame.

104. The kit of claim 100 wherein at least one frame is a femoral frame.

105. A bone milling device, comprising
a drive member;
a cutting means; and
a radial support means, wherein said radial support means projects along an outer circumference of said cutting means, and wherein said cutting means projects beyond said radial support means by a distance equal to a depth of a cut made by said cutting means.

106. The bone milling device of claim 105, wherein said radial support means partially circumscribes said cutting means.

107. The bone milling device of claim 105 wherein said radial support means fully circumscribes said cutting means.

108. A method of milling a bone, comprising the steps of
contacting a surface of said bone with a bone milling device comprising
a drive member,
a cutting means, and
a radial support means, wherein said radial support means projects along an outer circumference of said cutting means, and wherein said cutting means projects beyond said radial support means by a distance equal to a depth of a cut made by said cutting means; and
milling a portion of said bone by guiding said bone milling device along said surface of said bone.

109. A bone milling kit, comprising
a femoral bone milling device, comprising
a drive member,
a cutting means, and
a radial support means, wherein said radial support means projects along an outer circumference of said cutting means, and wherein said cutting means projects beyond said radial support means by a distance equal to a depth of a cut made by said cutting means; and
at least one femoral implant.

110. The bone milling kit of claim 109, further comprising
at least one tibial implant, at least one tibial template, and a milling device for use with said tibial template.

111. A bone milling apparatus, comprising
i) a cutting device comprising
a drive member,
a cutting means, and,
a chucking mechanism connecting said drive member to said cutting means; and
ii) a frame having a top surface and a bottom surface, said frame having a slot extending there through from said top surface to said bottom surface, and at least one support means disposed on said bottom surface;
wherein said chucking means extends through said slot, and wherein a bottom surface of said cutting means projects beyond said support means by a distance equal to a depth of a cut made by said cutting means.

112. The bone milling apparatus of claim 111, further comprising a latching mechanism to reversibly fix said cutting device at a position along said slot.

113. A method of milling a bone, comprising the steps of
contacting a surface of said bone with a bone milling apparatus, comprising
i) a cutting device comprising
a drive member,
a cutting means, and,
a chucking mechanism connecting said drive member to said cutting means; and
ii) a frame having a top surface and a bottom surface, said frame having a slot extending there through from said top surface to said bottom surface, and at least one support means disposed on said bottom surface;
wherein said chucking means extends through said slot, and wherein a bottom surface of said cutting means projects beyond said support means by a distance equal to a depth of a cut made by said cutting means; and
milling a portion of said bone surface by guiding said bone milling apparatus over said surface of said bone.

114. The method of claim 113, wherein said bone milling apparatus further comprises a latching mechanism to reversibly fix said cutting device at a position along said slot.

115. A bone milling kit, comprising,
a bone milling apparatus, comprising
i) a cutting device comprising
a drive member,
a cutting means, and,
a chucking mechanism connecting said drive member to said cutting means; and
ii) a frame having a top surface and a bottom surface, said frame having a slot extending there through from said top surface to said bottom surface, and at least one support means disposed on said bottom surface;
wherein said chucking means extends through said slot, and wherein a bottom surface of said cutting means projects beyond said support means by a distance equal to a depth of a cut made by said cutting means; and
iii) an implant.

116. The bone milling kit of claim 115 further comprising at least one tibial implant, at least one tibial template, and a milling device for use with said tibial template.

117. The bone milling kit of claim 115, wherein said bone milling apparatus further comprises a latching mechanism to reversibly fix said cutting device at a position along said slot.

118. The kit of claim 14 further comprising a plurality of tibial implants and at least one femoral implant.

119. The kit of claim 15 wherein said femoral frame is curved to match at least one curve of a femur bone.

120. The bone milling kit of claim 99 further comprising at least one implant.

121. The bone milling kit of claim 99, wherein said at least one frame is a plurality of frames.
